(19) United States
(12) Patent Application Publication
(10) Pub. No.: US 2024/0011679 A1
(43) Pub. Date: Jan. 11, 2024

(54) WEARABLE THERMAL MANAGEMENT DEVICE

(71) Applicant: 3M INNOVATIVE PROPERTIES COMPANY, St. Paul, MN (US)

(72) Inventors: Milind B. Sabade, Woodbury, MN (US); Ravi Palaniswamy, Singapore (SG); Ryan J. Rogers, Shoreview, MN (US); Diane L. Emslander, City of Grant, MN (US); Dominic J. Julian, Woodbury, MN (US)

(21) Appl. No.: 18/035,864

(22) PCT Filed: Dec. 2, 2021
(86) PCT No.: PCT/IB2021/061265

§ 371 Date: May 8, 2023

Related U.S. Application Data
(60) Provisional application No. 63/121,378, filed on Dec. 4, 2020.

(51) Int. Cl. F25B 21/02; H10N 10/17

(52) U.S. Cl. CPC F25B 21/02; H10N 10/17

(57) ABSTRACT

The present invention is a thermal management device including a power source, a flexible thermoelectric module, and a first phase change material.

### BACKGROUND

Phase change material (PCM) are substances which absorb or release sufficient amount of energy at phase transition to provide useful heating and cooling. Generally, the transition is from one of the first two fundamental states of matter, namely solid and liquid to the other. Most PCMs are reversible and can be used for heating or cooling depending on the temperature change. Due to their ability to store latent heat, PCMs can be used in various applications where thermal storage is desirable, including as personal heating and cooling devices. For example, PCMs are currently used in heating pads, support devices, and clothing.

Thermoelectric devices have also been widely used for heating or cooling purposes. Heat sinks (e.g., ceramic or metal plates) are used for managing heat on the hot side of the thermoelectric device.

### BRIEF DESCRIPTION OF THE DRAWINGS

This disclosure may be more completely understood in consideration of the following detailed description of various embodiments of the disclosure in connection with the accompanying drawings, in which:

FIG. 1 is a cross-sectional view of a first embodiment of a thermal management device of present invention.

FIG. 2 is a cross-sectional view of a second embodiment of a thermal management device of present invention.

FIG. 3 is a cross-sectional view of a third embodiment of a thermal management device of present invention.

FIG. 4 is a cross-sectional view of a fourth embodiment of a thermal management device of present invention.

FIG. 5 is a cross-sectional view of a fifth embodiment of a thermal management device of present invention.

FIGS. 6A and 6B are cross-sectional views of a sixth embodiment of a thermal management device of present invention.

FIG. 7 is a cross-sectional view of a seventh embodiment of a thermal management device of present invention.

FIGS. 8A and 8B are schematics of the seventh embodiment of the thermal management device of present invention.

While the above-identified figures set forth several embodiments of the disclosure, other embodiments are also contemplated, as noted in the description. In all cases, this disclosure presents the invention by way of representation and not limitation. It should be understood that numerous other modifications and embodiments can be devised by those skilled in the art, which fall within the scope and spirit of the principles of the invention.

### SUMMARY

In one embodiment, the present invention is a thermal management device including a power source, a flexible thermoelectric module, and a first phase change material.

In another embodiment, the present invention is a wearable thermal management device having an innermost layer positionable adjacent a wearer and an outermost layer. The wearable thermal management device includes a phase change material, a thermoelectric module; and a power source. The phase change material is the innermost layer.

### DETAILED DESCRIPTION

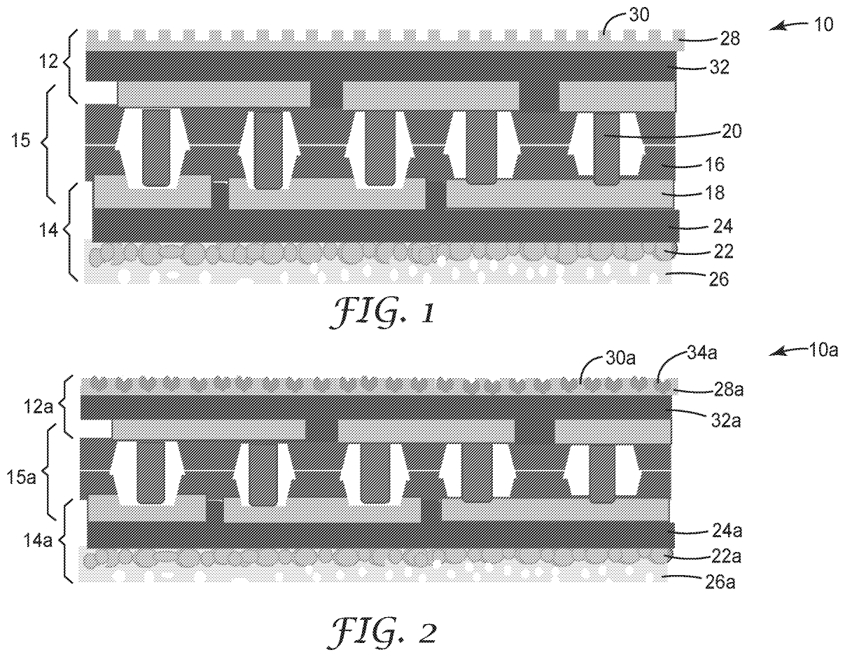

The present invention is a thermal management device that combines a flexible thermoelectric module and a phase change material (PCM) and is wearable. FIGS. 1-8B show various embodiments of the thermal management device which generally includes a phase change material, a flexible thermoelectric module, and a power source. The thermal management device may also optionally include other elements, such as heat fins and a conductive textile. The thermal management device is capable of being maintained within a desired temperature range using a reduced number of thermoelectric modules and energy required from a power source, such as a battery. The thermoelectric module of the thermal management device uses the power to either heat or cool the thermal management device to the desired temperature. When at the desired temperature, the phase change material functions to maintain the desired temperature by retaining latent heat/cooling storage within the phase change material. By combining the phase change material with the flexible thermoelectric module, the load of the flexible thermoelectric module is reduced. Thus, it is the combination of the phase change material and flexible thermoelectric module that allows the thermal management device to have a reduced number of thermoelectric modules and energy required from the power source to maintain the temperature of the device it is supporting.

Thermoelectric modules are solid-state having no moving parts and do not require any fluids for heating or cooling. The thermoelectric module can work as a heater or cooler, based on the Seebeck effect and the Peltier (reverse of Seebeck) effect, respectively. The thermoelectric module can work as a cooler or heater based on the so-called Peltier effect. When an electric current flows through the module, it brings heat from one side to the other so that one side gets cooler while the other gets hotter. A benefit of thermoelectric modules is that they can increase the efficiency of a system while decreasing the environmental impact by creating power from heat. The thermoelectric module takes advantage of temperature differentials in the system to generate power and is bi-directional, meaning that it can both heat and cool in the same device. In the thermal management device of the present invention, the thermoelectric module works in conjunction with the phase change material so that the thermal management device can provide constant cooling/heating capacity to deliver the heat or cool effectively while not draining the power source too quickly.

The thermoelectric module of the present invention is flexible in order to better conform to the thermal management module. In one embodiment, the flexible thermoelectric module is flexible enough to conform to or wrap around to an object surface having various shapes and angles. The thermoelectric module includes a flexible substrate having a plurality of thermoelectric modules or screen-printed thermoelectric ink supported by the flexible substrate. The thermoelectric modules are connected by electrodes. The substrate may be a flexible dielectric substrate made of any suitable materials such as, for example, polyimide, liquid crystalline polymers (LCP), polyester, poly ether ether ketone (PEEK) polymer, PDMS, polyaramid, other thermoplastic-based films, etc. The electrodes can include any suitable electrically conductive materials such as, metals, metal alloys, etc. The thermoelectric modules include one or more p-type thermoelectric modules and one or more n-type thermoelectric modules either in the form of semi-conductor chips or screen printable p- and n-type inks alternatingly connected in series by the electrodes.

In one embodiment, the thermoelectric module is in the form of a puck. In one embodiment, the puck has a weight of about 200 grams, a diameter of about 3 inches, and a thickness of about 0.5 inches or greater.

Phase change materials (PCMs) are materials with a high heat of fusion that, when melting or solidifying at a certain temperature (that is, undergoing a phase change), can store and release large amounts of energy. During a phase change (such as melting or solidifying or freezing), molecules rearrange themselves and cause an entropy change that results in the absorption or release of latent heat. Throughout a phase change, the temperature of the material itself remains constant and does not affect the temperature of the device or system it is in. The phase change material thus functions to absorb and release energy to maintain a constant temperature. The phase change material provides cushioning or enhanced comfort, improves uniformity/distribution of heat/cold, minimizes the amount of power used to power the thermoelectric module, and reduces the size of the thermoelectric module necessary for a given temperature profile.

The phase change material is also tunable, meaning that the phase change material can be tuned to specific temperatures. This property is needed for the phase change material to work in concert with either a heating or cooling profile at a specific temperature range to provide a desired therapy. The phase change material is also swappable in that if the phase change material cannot support both a hot and a cold temperature profile with the same phase change material, the phase change material can be switched out for cooling or heating based on its tuned properties. In one embodiment, the phase change material may be enclosed in a pouch so that it may be easily removable and replaceable within the wearable thermal management device. In one embodiment, when more than one phase change material is used, the phase change materials may be color coded to avoid confusion during use. For example, the phase change material may be colored blue for cooling and red for heating.

In one embodiment, the phase change material may be made of a sustainable material. For example, the phase change material is at least partially biodegradable or compostable. As used herein, a material is “compostable” when it is capable of breaking down into natural elements in a compost environment. As used herein, “compostable” refers to materials that undergo degradation by biological processes during composting to yield carbon dioxide, water, inorganic compounds, and biomass at a rate consistent with other compostable materials and leaves no visible, distinguishable or toxic residue. As used herein, “biodegradable” refers to materials or products that meet the requirements of ASTM D6400. In one embodiment, the phase change material may be made of a plant-based material, including, but not limited to, soybean or coconut oils.

The thermal management device also includes a power source. In one embodiment, the power source is a battery. In one embodiment, the battery may be flexible or conformable. The battery can be rechargeable using an external power source (e.g. wall outlet) but may also be capable of being disconnected from the external power source to allow the thermal management device to be mobile for an extended period of time, allowing the thermoelectric module to be powered solely by the battery. Various types of batteries can be used, as will be recognized by those of skill in the art. For example, the battery can be a standard battery or can be an organic, printable zinc poly battery for the purposes of sustainable disposal. The battery can include a USB or plug to allow it to be connected into the power source. In one embodiment, the battery and plug are swappable to ease replacement of the parts when needed. In one embodiment, the battery can be in the form of a pouch cell. In one embodiment, the battery has a weight of about 300 grams and is about 2 inches by 4 inches by 1 inch.

In another embodiment, the thermal management device is powered directly from power source, such as a wall outlet. This may provide the thermal management device to maintain designated temperatures for a longer period of time than if connected to a battery.

The thermal management device can optionally include a means for minimizing heat loss, and thereby further helping with the performance of the thermoelectric module. In one embodiment, the thermal management device includes an insulating material or a coating such as an insulative or low thermal conductivity material for energy harvesting. An example of a suitable insulating material includes, but is not limited to, a THINSULATE™ material sold by 3M Company, located in St. Paul, MN. Another example of a suitable insulator includes, but is not limited to, an aerogel. An aerogel is a low-density solid-state substance similar to a gel where the liquid component is replaced with gas. Aerogels have very low thermal conductivity.

To increase comfortability, in one embodiment, the thermal management device also includes a moisture control, or wicking capability. For example, a condensation management film may be used. This can be particularly useful if the thermal management device is used to provide therapy for wounds and traumas where direct skin contact with moisture or water condensation control is required. In another embodiment, the thermal management device may have the ability to wet the interface between the thermal management device and the skin of the wearer. Wetting the interface can help distribute the energy transfer as well as increase efficiency.

The thermal management device can also include integrated power electronics that allows modification of the target temperature, allowing the target temperature to be turned up or down. The target temperature can be controlled either manually or through a remote application. For example, the temperature may be remotely controlled through phone-based application. In one embodiment, the target temperature may be a specific temperature set by the user. In another embodiment, the target temperature may be variable to maintain the surface in contact with the thermal management device (i.e., the skin of the user) at a specific temperature. Thus, the thermal management device will raise or lower the temperature output to maintain a specific temperature at the surface contact.

In one embodiment, when the thermal management device is integrated into a wearable article, if the temperature control is manual, it can be located on the wearable article itself. If the temperature control is via a remote application, it can be controlled, for example, via Blue Tooth Low Energy (BLE) communication with a receiver in the wearable device. This allows the thermal management device to provide instant and on demand heating or cooling. One advantage of the thermal management device is that it can provide a desired level of cooling or heating and maintain that temperature for a desired length of time without the need to replace a cooling device or heating device at the desired intervals. For example, the thermal management device can be set to automatically provide a heating cycle for a predetermined amount of time, and then provide a cooling cycle for a predetermined amount of time. In one embodiment, the thermal management device can cycle for an extended duration, such as eight hours, to provide the desired heating and cooling at the desired intervals. The heating cycles can provide aid with pain while the cooling cycles can provide cold therapy to aid with swelling. In one embodiment, the thermal management device requires about 1 minute or less to cool down to about 35-45° F. from room temperature. In one embodiment, the thermal management device includes an auto shut off feature for safety and to prevent injury. The auto shut off can occur for example, after a predetermined amount of time, or if the temperature goes above or below a preset amount.

When the thermal management device is used as part of a wearable article, the phase change material is generally positioned as the innermost layer closest to the wearer. A softer or more conformable phase change material can be chosen such that it provides cushioning and comfort to the user of the thermal management device. In one embodiment, the power source is positioned as the outermost layer relative to the wearer in order to interconnect with the thermoelectric module, positioned in direct contact with the phase change material layer. In another embodiment, the power source is positioned between the phase change material and the thermoelectric module. In another embodiment, the thermal management device also includes an insulator. When the thermal management device includes an insulator, the insulator can be positioned as the outermost layer or positioned between the thermoelectric module and the battery.

FIG. 1 shows a cross-sectional view of a first embodiment of the thermal management device 10 of the present invention having a hot side 12 and a cold side 14. The thermal management device 10 includes the flexible thermoelectric module 15 having a dielectric layer 16, copper (Cu) layer 18, and TEG chips 20 between the hot side 12 and cold side 14. In the embodiment shown in FIG. 1, a first phase change material layer 22 is positioned on the cold side 14 along with a first high conductive thermal interface material 24 and aides in maintaining the temperature at the cold side 14. In one embodiment, the first phase change material is kept in a porous flexible pouch or non-woven material 26. The hot side 12 of the management device 10 is attached with a thin and conformable heat sink 28 having heat fins 30. The heat sink 28 is attached using a second high conductive thermal interface material 32. In one embodiment, the high conductive thermal materials 24 and 32 include two-part conductive epoxy adhesives/high conductive thermal interface materials, a conductive thermal pad, and a conductive pressure sensitive adhesive.

FIG. 2 shows a cross-sectional view of a second embodiment of the thermal management device 10a. The second embodiment of the thermal management device 10a is similar to the first embodiment of the thermal management device 10 except that in addition to a first phase change material 22a positioned at the cold side 14a, a second phase change material 34a is positioned adjacent to the heat sink 28a at the hot side 12a. Thus, the first phase change material 22a helps maintain the temperature on the cold side 14a and the second phase change material 34a helps maintain the temperature on the hot side 12a. The combination of the second phase change material 34a along with heat fins of the heat sink 28a provides quick and uniform heat spreading, which enhances the performance of the hot side 12a of the thermal management device 10a.

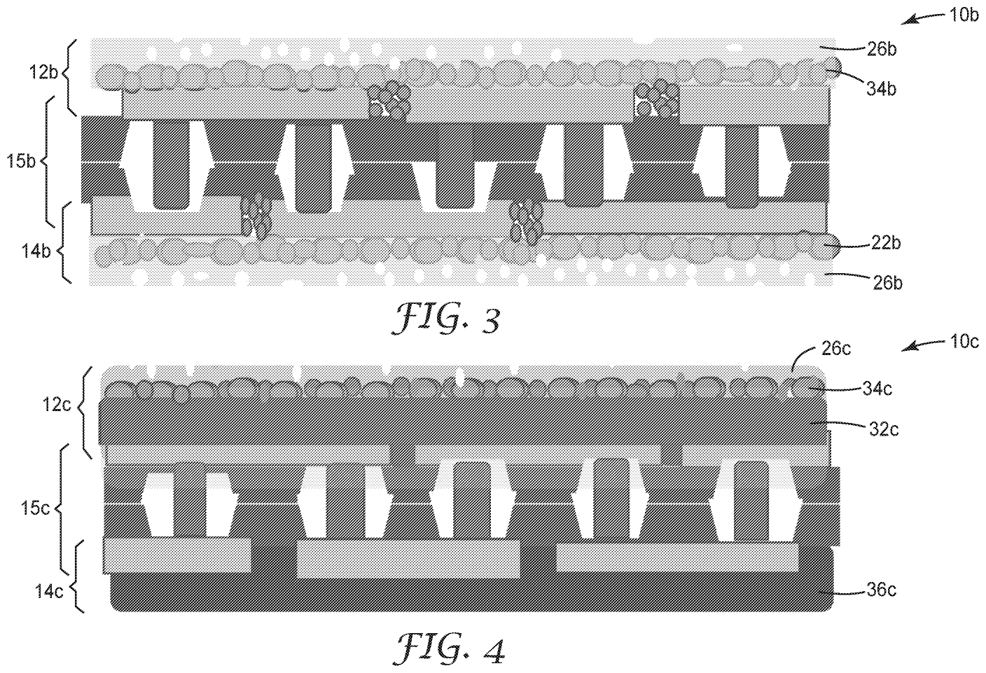

FIG. 3 shows a cross-sectional view of a third embodiment of the thermal management device 10b which includes a first and second phase change material 22b and 34b on the cold and hot sides 14b and 12b, respectively, of the thermal management device 10b. The first and second phase change materials 22b and 34b have different thermal characteristics and reverse phenomenon. The third embodiment of the thermal management device 10b does not include thermal interface materials or a heat sink but includes porous flexible pouches or non-woven materials 26b to maintain the phase change materials 22b and 34b.

FIG. 4 shows a cross-sectional view of a fourth embodiment of the thermal management device 10c of the present invention. In the embodiment shown in FIG. 4, the thermal management device 10c includes a thermal conductive cushioning pad or thermal conductive textile cushioning material 36c on the cold side 14c of the thermal management devices 10c. The hot side 12c of the thermal management device 10c includes a thermal interface material 32c and phase change material 34c (with or without the conformable heat fins) within a porous flexible pouch or non-woven material 26b for thermal management of the hot side 12c of the thermal management device 10c.

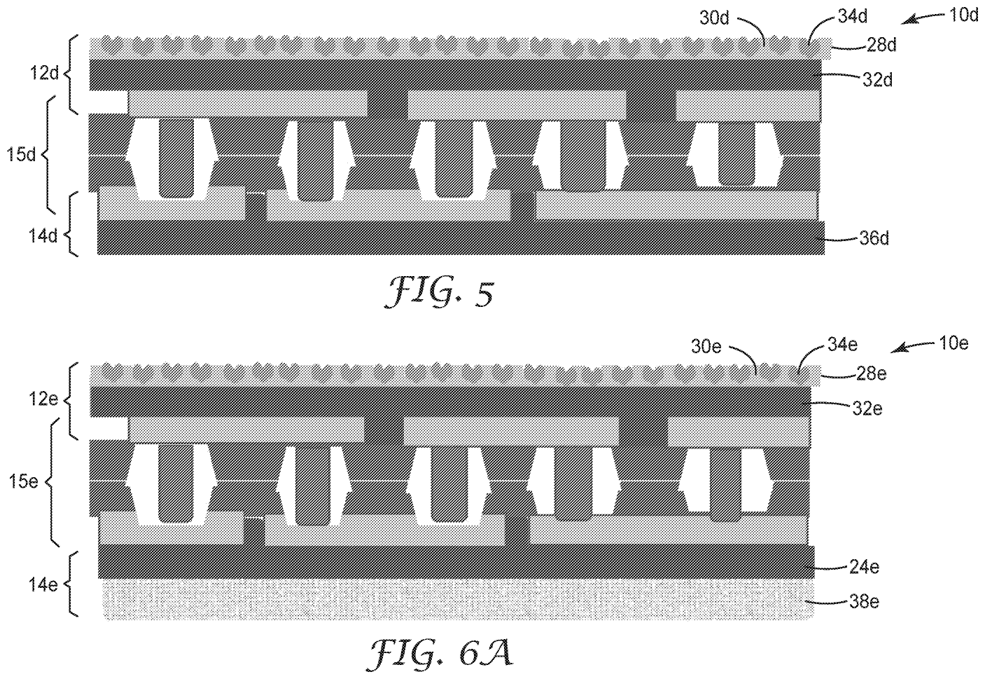

FIG. 5 shows a cross-sectional view of a fifth embodiment of the thermal management device 10d of the present invention. In the fifth embodiment, the thermal management device 10d includes a thermal interface material 32d and phase change material 34d along with flexible heat fins 30d for thermal management on the hot side 12d. The cold side 14d includes a thermal conductive cushioning pad 36d.

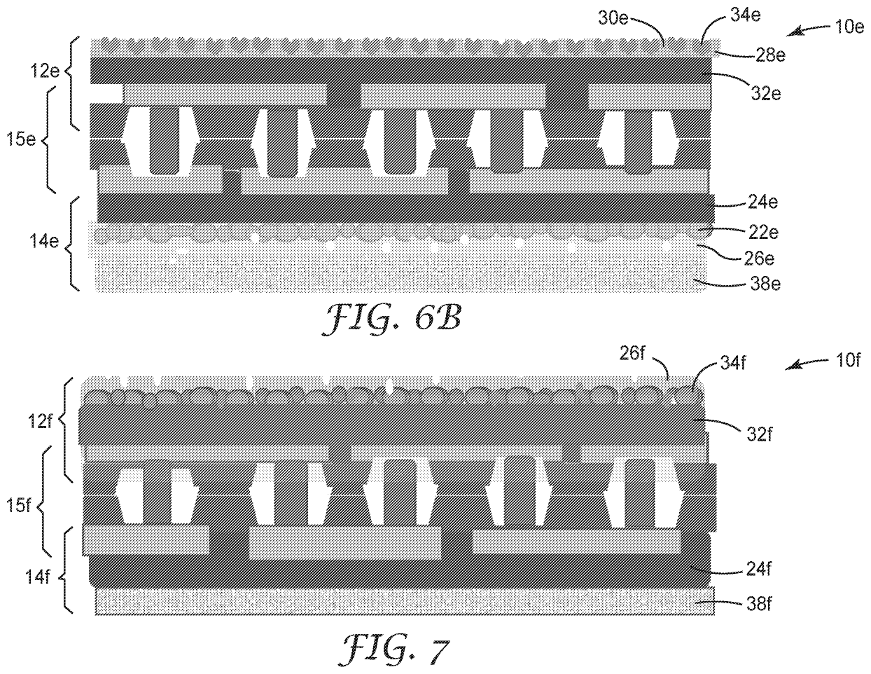

FIGS. 6A and 6B show a cross-sectional view of a sixth embodiment of the thermal management device 10e which is similar to the fifth embodiment except that a conductive textile 38e is added to the cold side 14e in the embodiment shown in FIG. 6A and a phase change material 22e, porous flexible pouch or non-woven material 26e, and conductive textile 38e is added to the cold side 14e in the embodiment shown in FIG. 6B. In addition, the cold side 14e includes thermal interface material 36e rather than a high conductive cushioning material. In one embodiment, the conductive textile includes a nano particle coating or extruded conductive fiber, etc. which can aid in transporting various information from the wearer. The phase change material 22e on the cold side 14e helps to provide a constant, uniform temperature on the cold side 14e. This facilitates the conductive textile 38e in gathering information using the temperature difference between the phase change material and the wearer.

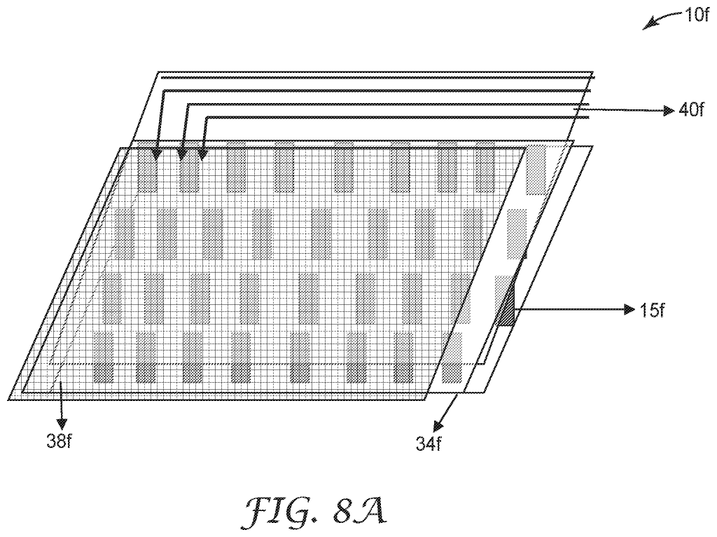

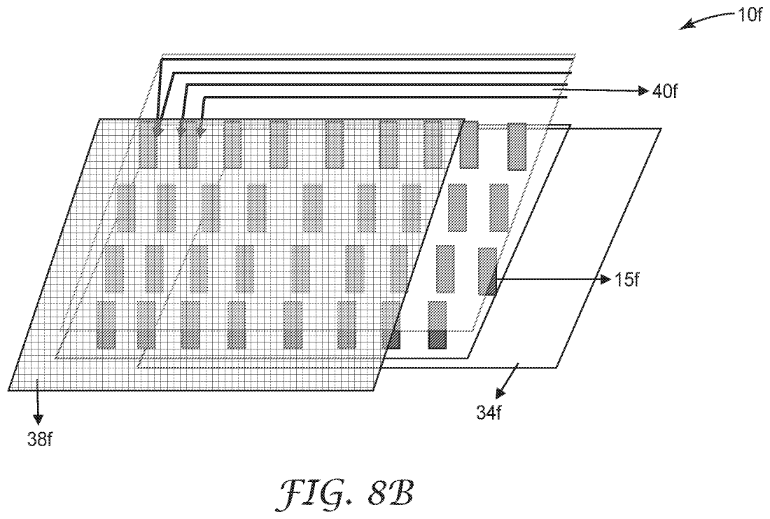

FIG. 7 shows a cross-sectional view and FIGS. 8A and 8B show schematics of a seventh embodiment of the thermal management device 10f which is similar to the fourth embodiment except that the cold side 14f of the seventh embodiment includes a thermal interface material 36f rather than a high conductive cushioning material. In addition, the seventh embodiment includes a thermal conductive textile 38f on the cold side 14f and a separate signal line 40f in the flexible thermoelectric module 15f (shown in FIGS. 8A and 8B). In one embodiment, the signal lines 40f are built using an electroplating process during the circuit making process of the cold side 14f of the flexible thermoelectric circuit. These signal lines 40f are directly connected with the conductive textile 38f on the cold side 14f and assist in transporting the signals generated by the conductive textile.

In one embodiment, the thermal management device is designed to be integrated into an article that allows it to be wearable while being flexible enough to conform to or wrap around an article having various shapes and angles. For example, the wearable article can be conformable around a body part such as a person's face, knee, shoulder, etc. as needed to provide comfort, fit, and efficient use of the heating or cooling capabilities. Because the thermal management device can be conformable, the user will experience increased comfort during use and can be used while the user is stationary, for example while sitting, or while the user is active. Uses of the thermal management device include, but are not limited to, support devices, such as wraps and braces. Integration of the thermal management device into a wrap or brace can provide a therapeutic effect by heating or cooling the joints.

In one embodiment, the thermal management device is integrated into a support device such as a brace. The brace may include, without limitation: a back brace, a neck brace, a shoulder brace, a wrist brace, an elbow brace, a finger brace, a knee brace, or an ankle brace. The brace can include a pocket into which the system can be positioned as an insert. The thermal management device is thus removable such that the brace can be worn with or without the thermal management device. By being removable, the brace is washable and is capable of lasting for an extended period of time. However, because the thermal management device can be easily integrated into the brace, the brace can function as an on the go cooling brace when desired. In one embodiment, the wearable article can be a lightweight cooling brace, or further, a cooling brace that provides cryotherapy.

While the specification primarily discusses the thermal management device as being used in conjunction with a support or brace, the thermal management device can be used in various other applications and devices to provide heating or cooling to a body. For example, the thermal management device can be used in helmets, earmuffs, air respirator systems, etc. The thermal management device can be used in a variety of applications where it may be desirable to maintain a temperature range without the need for other components such as gel packs or electrical wires to power up the system. In addition, because the thermal management device can be cordless, it is mobile and easily transportable. In one embodiment, the thermal management device has wireless connectivity via wireless cellular or non-cellular networks. In one embodiment, the thermal management device allows for remote monitoring via a web-based or application-based application. This can be used, for example, to monitor an injury or trauma via telemedicine (i.e., via remote monitoring interface). In an embodiment such as this, the thermal management device can incorporate a microcontroller for data generation, data analysis, and for remote patient monitoring. Having this information can also allow for data collection, data analytics, insights, and/or recovery profile to be used to carve a path forward for future treatment options. In this type of configuration, the body can function as a sensor or as an interface with the signal transmitting/receiving device.

When used as a wearable technology, the thermal management device can be combined (via, for example, co-weaving or meshing) with an e-fabric with nanomaterial coated textiles to keep the body warm or cool. Skin emits infrared radiation in a specific range of wavelengths. By manipulating the ways in which such e-sensor based textile fabrics block or transmit radiation in this band, multiple textiles can have different effects on temperature. For example, to heat the body, metallized polyethylene textile can minimize heat radiation loss while still being breathable. Compared with normal textiles, nanotextiles can keep bodies about 7° C. warmer whereas under direct sunlight the cooling fabric, a novel nanocomposite material, can cool the body by more than 10° C.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. In particular, as used herein, the recitation of numerical ranges by endpoints is intended to include all numbers subsumed within that range (e.g., 1 to 5 includes 1, 1.5, 2, 2.75, 3, 3.80, 4, and 5). In addition, all numbers used herein are assumed to be modified by the term “about.” Furthermore, various exemplary embodiments have been described. These and other embodiments are within the scope of the following claims.

What is claimed is:

1. A thermal management device comprising:
a power source;
a flexible thermoelectric module; and
a first phase change material.

2. The thermal management device of claim 1, further comprising an insulator.

3. The thermal management device of claim 2, wherein the insulator is an aerogel material.

4. The thermal management device of claim 1, wherein the first phase change material can also function as a cushion.

5. The thermal management device of claim 1, further comprising a second phase change material.

6. The thermal management device of claim 5, wherein the first phase change material and second change material are swappable.

7. The thermal management device of claim 1, wherein the first phase change material is enclosed within a pouch.

8. The thermal management device of claim 1, further comprising a thermal interphase material.

9. The thermal management device of claim 1, further comprising a conformable heat sink.

10. The thermal management device of claim 9, wherein the conformable heat sink includes a conformable heat fin.

11. The thermal management device of claim 10, wherein the first change phase material surrounds the heat fin.

12. The thermal management device of claim 1, wherein the first phase change material is tunable.

13. The thermal management device of claim 1, wherein the thermal management device is wearable.

14. A wearable thermal management device having an innermost layer positionable adjacent a wearer and an outermost layer, the wearable thermal management device comprising:
a phase change material, wherein the phase change material is the innermost layer;
a thermoelectric module; and
a power source.

15. The wearable thermal management device of claim 13, wherein the phase change material functions as a cushion.

16. The wearable thermal management device of claim 13, further comprising an insulator.

17. The wearable thermal management device of claim 15, wherein the insulator is an aerogel.

18. The wearable thermal management device of claim 13, wherein the insulator is positioned adjacent the power source.

19. The wearable thermal management device of claim 13, wherein the power source is a battery.

20. The wearable thermal management device of claim 13, wherein the wearable thermal management device is positioned within a brace or support.
